(19) United States
(12) Patent Application Publication
(10) Pub. No.: US 2025/0339019 A1
(43) Pub. Date: Nov. 6, 2025

(54) Surgical Tool With Proximally Mounted Camera For Use With Cannulas

(71) Applicant: Viseon, Inc., Irvine, CA (US)

(72) Inventors: Todd D. McIntyre, Irvine, CA (US); Peter G. Davis, Irvine, CA (US)

(73) Assignee: Viseon, Inc., Irvine, CA (US)

(21) Appl. No.: 18/975,771

(22) Filed: Dec. 10, 2024

Related U.S. Application Data
(60) Provisional application No. 63/642,258, filed on May 3, 2024.

(51) Int. Cl. A61B 1/06; A61B 1/015; A61B 1/04

(52) U.S. Cl. CPC A61B 1/0684; A61B 1/015; A61B 1/042

(57) ABSTRACT

The devices and methods for visualization of a surgical workspace in the brain while using a surgical tool such an aspirator. A tool is fitted with a camera assembly, at a position at the proximal end of an insertion portion and distal tip of the tool. Illumination is provided through a light guide (a cylindrical light guide or bundle of optical fibers) which terminates proximal to the distal tip of the tool. The camera assembly is aimed to intersect the long axis of the tube near the distal tip. The camera is disposed on the proximal end of the tool such as it resides outside the body when the tool tip is disposed in the surgical workspace, while providing images of tissue in the workspace while a surgeon manipulates the tool within the surgical workspace.

The application claims priority to U.S. Provisional Application 63/642,258, filed May 5, 2024.

### FIELD OF THE INVENTIONS

The inventions described below relate to the field of minimally invasive surgery.

### BACKGROUND

Systems for visualization of a surgical space within the body, using a camera mounted on a surgical tool, have been disclosed. Shahinian, Endoscope System and Method of Operation Thereof, U.S. Pat. No. 9,861,261 (Jan. 9, 2018) discloses a “hybrid” endoscopic tool for use in surgery which includes a camera mounted on the distal end of an endoscopic instrument. The camera is mounted on the side of the instrument, near the distal end of the instrument, such that it resides within the body and surgical workspace during use. Lighting in this system is provided by a light ring which is coterminous with the tool. While useful in surgeries with large surgical workspaces, this is unsuitable for use in the brain where there is no empty space or voids in the tissue, and twisting and longitudinal translation of the device would disrupt brain tissue pressing against the tool and its camera.

### SUMMARY

The devices and methods described below provide for visualization of a surgical workspace in the brain while using a surgical tool such as an aspirator. A tool is fitted with a camera assembly, at a position at the proximal end of an insertion portion and distal tip of the tool. Illumination is provided through a light guide (a cylindrical light guide or optical fiber bundle) which terminates proximal to the distal tip of the tool. The camera assembly is aimed to intersect the long axis of the tube near the distal tip. The camera is disposed on the proximal end of the tool such that it resides outside the body when the tool tip is disposed in the surgical workspace, while providing images of tissue in the workspace while a surgeon manipulates the tool within the surgical workspace. The system can be manufactured with tools of varying lengths, while using a camera assembly of the same geometry for the tools of varying lengths, and the camera assemblies may be rotating to point the viewing axis toward the tip and thereafter fixed.

### BRIEF DESCRIPTION OF THE DRAWINGS

FIG. 1 illustrates a system comprising a surgical tool, such as an aspirator, with a proximally mounted camera, in a cannula.

FIG. 2 is a cross-section of a surgical tool according to FIG. 1, showing additional features.

FIG. 3 is a cross-section of a surgical tool according to FIG. 1, showing the fluid flow path and cooling features.

FIG. 4 is a perspective view of the surgical tool.

FIG. 5 illustrates a mirror adapter for use with the surgical tool.

### DETAILED DESCRIPTION OF THE INVENTIONS

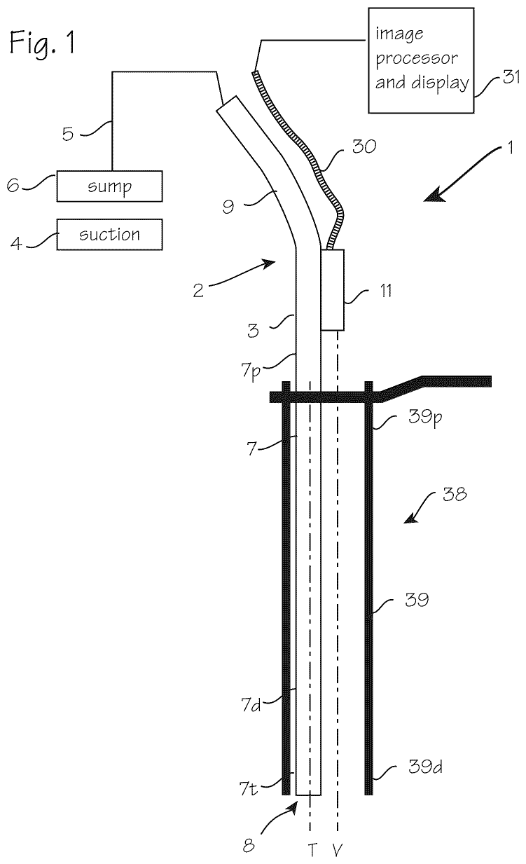

FIG. 1 illustrates surgical system 1 including an aspirator system 2 comprising an aspirator tube 3 and a vacuum source 4 connected to the aspirator tube 3 through a suction line 5 and a sump 6 for collection of any tissue collected through the aspirator tube 3. The aspirator tube 3 comprises an insertion portion 7 which typically consists of a slender tube with distal portion 7d configured for insertion into a surgical workspace and a proximal portion 7p which typically remains outside the surgical workspace (outside the body of a patient) during use. A suction port 8 is located at or very near the distal tip 7t of the distal portion 7d. Preferably, the aspirator tube also comprises a connection portion 9 extending proximally from the insertion portion, and this connection portion may be offset from the straight insertion portion 7 at an angle so that the aspirator tube may be held at a point offset from the straight insertion portion 7, so that the surgical workspace is not obscured by the surgeon's hand or other manipulator during use. The straight insertion portion 7 of the aspirator tube is characterized by a longitudinal axis T, and is preferably configured for insertion into a surgical workspace through a cannula (straight enough and long enough to reach a surgical workspace at the distal end of a cannula with which it is to be used.)

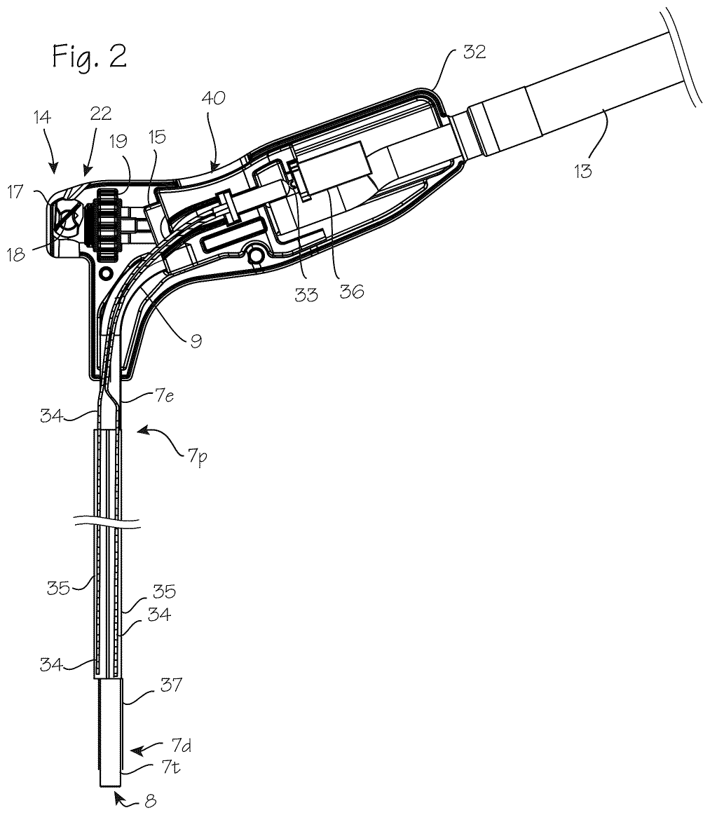

The surgical system includes a camera assembly 12 mounted on the proximal portion 7p of the straight insertion portion 7 in this illustration (FIG. 2 illustrates an alternative placement). The camera assembly is characterized by a viewing axis V, which may be aligned with the axis T of the straight insertion portion 7 of the aspirator tube, substantially parallel to the long axis or intersecting the long axis at a point near the distal end of the aspirator tube. Alternatively, the camera assembly may be mounted proximally to the straight insertion portion 7, on or above the offset connection portion 9 (as in FIGS. 2-4), in which case the camera assembly may include prisms or mirrors that may be located on the proximal portion 7p so as to locate the viewing axis parallel to the axis T of the straight insertion portion 7 (see FIG. 2). The distal most optical component, which may be an optical surface (a lens, filter, reflector (prism or mirror)), is located proximally relative to the straight insertion portion 7, well-spaced from the distal portion 7d, so that it does not block view of the surgeon into the surgical space around the distal tip 7t.

In summary, FIG. 1 depicts an aspirator system for aspirating tissue from a surgical workspace within a patient's body. The system includes an aspirator tube and a vacuum source connected to the aspirator tube through a suction line. The aspirator tube includes an insertion portion configured for insertion into the surgical workspace through a cannula, said aspirator tube having a distal end and a proximal end and a longitudinal axis, with an aspiration port disposed proximate the distal end. The system also includes a camera assembly mounted on the proximal end of the aspirator tube. The camera has a viewing axis aligned with the long axis of the aspirator tube, and a distal-most optical surface which may be an objective lens, prism, or optical filter. The system also includes a cannula which includes a cannula tube with an open distal end and an open proximal end, and a lumen extending from said distal end to said proximal end. The aspirator tube is longer than the lumen of the cannula, such that, with the aspirator tube disposed within the lumen of the cannula and the distal end of the aspirator tube disposed proximate the tissue to be aspirated, the distal-most optical surface of the camera assembly is disposed outside the proximal end of the cannula.

The system has been described in reference to an aspirator. The imaging system arrangement may be used in combination with other tools, such as a dissector, an ablation probe, a macerator, or any other elongate tool configured for use within a cannula. Thus, a surgical tool configured for insertion into the surgical workspace through a cannula may include a camera assembly mounted on the proximal end of the tool, with the viewing axis aligned with the long axis of the tool, and a distal-most optical surface which may be an objective lens, prism, or optical filter, with a cannula which includes a cannula tube with an open distal end and an open proximal end, and a lumen extending from said distal end to said proximal end. As mentioned in reference to the aspirator tube, the tool is longer than the lumen of the cannula, such that, with the tool disposed within the lumen of the cannula and the distal end of the tool (including an electrode, macerating structure, etc.) disposed proximate the tissue proximate the distal opening of the cannula, the distal-most optical surface of the camera assembly is disposed outside the proximal end of the cannula.

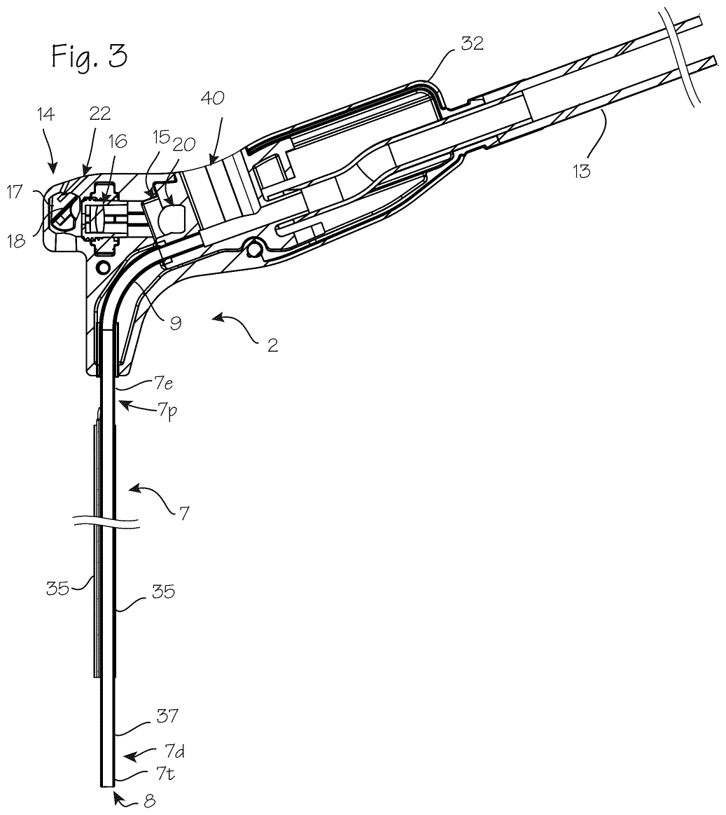

FIGS. 2 and 3 are cross-sections of a surgical tool similar to the tool of FIG. 1, showing additional features. As shown in FIG. 2, the camera assembly 12 comprises a housing 14 (which may be integral with the remainder of the housing 32) with an imaging sensor 15 disposed at a first end of the housing, a lens assembly 16, a rotatable reflector mount 17 and a reflector 18 mounted on or within the reflector mount at the second end of the housing. The rotatable reflector mount is rotatably disposed within the housing such that it can tilt the mirror in planes intersecting the central viewing axis of the imaging sensor (typically perpendicular to the flat face of the imaging sensor). The reflector establishes a central viewing axis V of the camera assembly, and the rotatable reflector mount provides a means for rotating the mirror and thereby altering the viewing axis relative to the imaging axis of the sensor to provide views of the surgical workspace at the distal end of the retractor blades or retractor tube. The lens assembly 16 is disposed within a focusing knob 19 which is rotatably mounted within the housing and operable to translate the lens assembly toward and away from the imaging sensor (and the reflector) to focus the system, with a portion of the knob exposed to the exterior of the housing so that it may be operated by a user. If desired for a compact construction, the imaging sensor 15 may be side-mounted and an additional reflector 20 may be used to direct images toward the imaging sensor.

The reflector mount may be rotated by a surgeon using the device, while using the device, to aim the camera toward the workspace at the distal end 7d of the insertion portion 7 using a post secured to the reflector mount. The rotatable reflector mount may be rotated to aim the camera toward the workspace at the distal portion 7d to intersect a long axis T of the aspirator tube 3 and thereafter may be rotationally fixed during manufacture, using a pin inserted through aperture 22.

The reflector mount may be rotatable only during the manufacturing process, and may be rotationally fixed relative to the housing during assembly to become a fixed reflector mount. The reflector mount may be rotationally fixed to point the viewing axis to intercept the long axis of the tube 3 in any manner, including use of a set screw through the aperture 22 or injection of fixing agent such as an adhesive, epoxy or melted plastic between the reflector mount and the surrounding housing. Use of a rotatable reflector mount and its fixation during manufacture (rather than intraoperatively) allows for proper aiming for aspiration tubes of different lengths without the need for camera assemblies of varying geometries, thereby simplifying the manufacture of tools of different lengths.

The camera assembly is connected through a video cable 30 to an image processor and video display 31.

The insertion portion of the aspirator tube is secured to a housing 32 which can serve as a handle for a user. A light source (lamp or LED 33 along with appropriate driver 36) is disposed within the housing, and a light guide (optical fibers, for example) 34 runs from the light source, along the aspirator tube, and are secured within a sheath 35. The LED driver 36 necessary for operation of the LED may also be disposed within the housing. The light guide (typically a bundle of optical fibers) originates proximate the LED and runs along the aspirator tube, under the sheath 35, and terminates distally, at a point well proximal to the distal end of the aspirator tube, in order to provide illumination to the surgical space while the distal tip 7t of the aspirator tube is disposed within tissue. Thus, the sheath also terminates at a point proximal to the distal tip 7t of the aspirator tube. The light guide is configured to emit light at various positions about the circumference of the aspirator tube, to provide illumination without shadows. The sheath may also terminate proximally at a point distal to the housing, or may have a gap in the proximal portion 7p leaving a proximal portion 7e of the aspirator tube exposed so that, in embodiments in which the aspirator tube is comprised, the aspirator tube may be touched with an RF electrode to transmit RF energy to tissue proximate the exposed distal tip 7t. An electrically insulative coating or sheath 37 may be applied over most of the distal portion 7d, excepting the distal tip 7t, to limit the area of the distal portion capable of transmitting RF energy into the tissue proximate the distal portion 7d. This additional sheath is configured with a small diameter, and may comprise an electrically insulative coating on an electrically conductive aspirator tube, so as not to obstruct light from the distal ends of the optical fibers or light guide.

To summarize the lighting features of the device shown in FIG. 2, the aspirator tool includes an aspirator tube and a vacuum source connected to the aspirator tube. The aspirator tube has an insertion portion configured for insertion into the surgical workspace through a cannula, with an aspiration port disposed proximate the distal end. The tool includes the housing with a distal end fixed to the proximal end of the aspirator tube and a proximal end fixed to a suction tube in fluid communication with a vacuum source. The housing has a longitudinal axis offset from the longitudinal axis of the aspirator tube, so that the housing may be used as a handle which does not block a surgeon's view of the cannula or the tissue visible through the lumen of the cannula. The camera assembly is mounted on the distal end of the housing, proximate the proximal end of the aspirator tube, with the camera a viewing axis aligned with the long axis of the aspirator tube. The LED light source is disposed within the housing and aligned to emit light into the light guide, which is disposed about the aspirator tube and extends along the aspirator tube from the proximal end of the aspirator tube toward the distal end of the aspirator tube. The light guide terminates distally at a point proximal to the aspiration port such that the light guide emits light through the lumen of the cannula to illuminate surfaces of tissue visible to the camera while the aspiration port is disposed in tissue near or beyond the distal opening of the cannula. The camera assembly has a distal-most optical surface, which is preferably disposed above (proximally displaced from) the proximal end of a cannula in which the aspirator tube is used, when the distal tip (and aspirator port) of the aspirator tube is near or beyond the distal opening of the cannula.

The surgical system may be used within a cannula 38 with a cannula tube 39 configured for insertion into the body of a patient, such that the distal end 39d is located within the body with the open distal end of the tube near a surgical workspace and the open proximal end 39p. The aspirator 3 is configured such that, with the distal portion 7d disposed within the cannula, and the distal tip 7t proximate the distal opening of the cannula tube and/or extending distally from the distal end 39d and/or into the surgical workspace, the proximal portion 7p of the insertion portion 7 extends proximally out of the cannula tube and the camera assembly, including the distal most component, is disposed proximally of the open proximal end 39p of the cannula tube 39. As appears from FIG. 1, the insertion portion 7 and/or aspirator tube 3 are sized such that, when the distal end 7d, distal tip 7t or suction port 8 of the insertion portion is disposed proximate the distal end 39d of the cannula tube, or disposed in tissue distally beyond the distal end 39d of the cannula tube, the distal-most element of the camera assembly is disposed proximal to the proximal end 39p of the cannula tube (there are no optical components of the camera within the cannula tube). The distal-most element of the camera assembly may be an objective lens, a prism, or the end of a light guide or optical fiber operably connected to the imaging sensor.

Within the housing the aspirator tube 3 is in fluid communication with the suction line 5, which is connected to the sump 6 and vacuum source 4. A suction control vent 40 in the housing provides for control of the application of suction after the vacuum source 4 has been turned on. With the suction control vent left unobstructed, the vacuum draws air into the handle, through the suction control vent 40, into the suction tube, and hence to the sump. The electronic components may be exposed to this airflow, or may be placed in direct contact with or close proximity to a portion of the suction tube such that heat from the electronic component is conducted to the suction tube and air flowing within the suction tube. This airflow through the handle and suction tube serves to cool electronic components housed within the housing, including the LED 33 and a driver circuit, which in turn allows use of higher power LED's within the housing (which is preferably configured for use as a handle of the tool). The excess heat of the components may be passed to the surrounding air or the user's hand.

To summarize the cooling features of the device shown in FIG. 2 the aspirator tool includes an aspirator tube and a vacuum source in fluid communication with the aspirator tube through a suction line, and, with both the aspirator tube and suction line communicating through the interior of a housing. The housing has a distal end fixed to the proximal end of the aspirator tube and a proximal end fixed to the suction line, and an outer wall, an interior space, and a suction control vent in the outer wall providing for fluid communication into the interior volume of the housing when the vent is unobstructed. An LED light source is disposed within the housing, aligned to emit light into a light guide configured to conduct light along the axis of the aspirator tube, and an LED driver is also disposed within the housing. To provide cooling to these electronic components, air flow over the components, or through a tube in thermal contact with these components, (1) the suction tube may be open to the interior volume of the housing, such that, when the vacuum source is operating and the suction port is at least partially unobstructed, ambient air flows into the housing and thence into the suction line, passing over (directly or indirectly) the electronic components or (2) the suction tube communicates with the control vent, and flow of ambient air through the control vent and through the suction tube provides cooling for the LED and LED driver, one or both of which are in thermal contact with the suction line.

The benefits of cooling the electronic components may be achieved alone or in combination with the benefits of the lighting features of FIG. 2 or the proximal camera arrangement of FIG. 1. Likewise, the benefits of the proximal camera arrangement of FIG. 1 can be achieved alone or in combination with the cooling features of FIG. 2 or the lighting features of FIG. 2, and the benefits of the lighting features of FIG. 2 can be achieved alone or in combination with the proximal camera arrangement of FIG. 1 or the cooling features of FIG. 2.

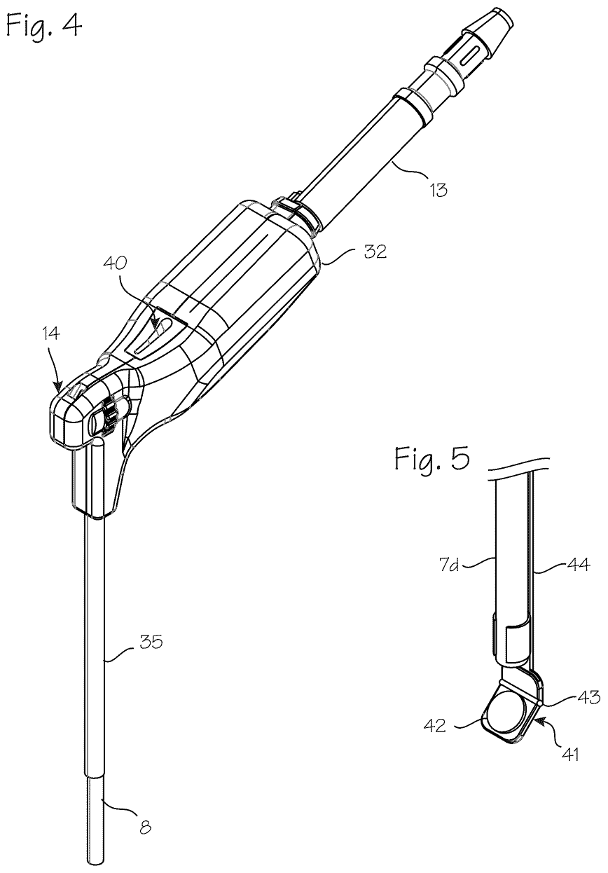

FIG. 4 is a perspective view of the surgical tool. This view shows the control suction control vent 40 communicating through a wall of the housing and further into. the section tube. With the control vent obstructed, all suction from the vacuum source 4 is applied to the aspirator tube. With the control vent unobstructed, the suction on the aspirator tube is weakened so little effective suction is applied to the tissue proximate the distal tip of the aspirator tube.

FIG. 5 illustrates a mirror assembly 41 and mirror 42 for use with the surgical tool. The mirror adaptor may be releasably fixed to the distal end 7d of the aspirator tube 7, with a surface oriented radially outwardly relative to the long axis T of the aspirator tube, so as to reflect images of tissue located to the side of the aspirator tube (angled from the long axis) to the reflector of the camera assembly. This mirror may be used to view tissue hidden behind structures in the brain, for example tissue located behind an aneurysm. The mirror may be rotatable about the distal end 7d of the aspirator tube insertion portion 7, to enable aiming the mirror without rotating the entire instrument, and may also be rotatable about a hinge to enable aiming the mirror up and down relative to the long axis of the device. The mirror assembly may include a hinge 43 to allow up and down rotation, and may include a rod 44 extending from mirror to the proximal end of the device to enable rotation up and down and rotation around the circumference of the distal end 7d.

In use, a surgeon will install the cannula in a surgical opening or anatomical opening in the patient, insert the insertion portion 7 of the aspirator tube 3 into the cannula so that the distal tip 7t and suction port 8 are with the surgical workspace, proximate tissue in the body (such as the brain). The surgeon will operate the camera assembly, image processor and display to obtain video images of the workspace near the distal tip and display those images on the display. The surgeon may then operate the aspirator to collect tissue from the workspace, using the video images from the camera to guide the distal tip 7t within the surgical workspace and collect tissue.

The aspirator and camera can be used with bladed retractors, or without retractors, employing the methods described above. While the invention has been illustrated within a system for aspiration, other surgical tools may be fitted with a proximally mounted camera and used as described above.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised.

We claim:

1. An aspirator system for aspirating tissue from a surgical workspace within a patient's body, said system comprising:
an aspirator tube and a vacuum source connected to the aspirator tube through a suction line, said aspirator tube comprising an insertion portion configured for insertion into the surgical workspace through a cannula, said aspirator tube having a distal end and a proximal end and a longitudinal axis, with an aspiration port disposed proximate the distal end;
a camera assembly mounted on the proximal end of the aspirator tube, said camera having a viewing axis aligned with the long axis of the aspirator tube, said camera assembly having a distal-most optical surface;
a cannula comprising a cannula tube characterized by an open distal end and an open proximal end, and a lumen extending from said distal end to said proximal end; wherein
the aspirator tube is longer than the lumen of the cannula, such that, with the aspirator tube disposed within the lumen of the cannula and the distal end of the aspirator tube disposed proximate the tissue to be aspirated, the distal-most optical surface of the camera assembly is disposed outside the proximal end of the cannula.

2. An aspirator system for aspirating tissue from a surgical workspace within a patient's body, said system comprising:
an aspirator tool comprising:
an aspirator tube and a vacuum source connected to the aspirator tube through a suction line, said aspirator tube comprising an insertion portion configured for insertion into the surgical workspace through a cannula, said aspirator tube having a distal end and a proximal end and a longitudinal axis, with an aspiration port disposed proximate the distal end;
a housing with a distal end fixed to the proximal end of the aspirator tube and a proximal end fixed to a suction tube in fluid communication with a vacuum source, said housing having a longitudinal axis offset from the longitudinal axis of the aspirator tube,
a camera assembly mounted on the distal end of the housing, proximate the proximal end of the aspirator tube, said camera having a viewing axis aligned with the long axis of the aspirator tube;
an LED light source disposed within the housing, aligned to emit light into a light guide disposed about the aspirator tube and extending along the aspirator tube from the proximal end of the aspirator tube toward the distal end of the aspirator tube, said light guide terminating distally at a point proximal to the aspiration port such that the light guide emits through the lumen to illuminate surfaces of tissue visible to the camera while the aspiration port is disposed in tissue.

3. An aspirator tool for aspirating tissue from a surgical workspace within a patient's body, said aspirator tool comprising:
an aspirator tube and a vacuum source connected to the aspirator tube through a suction line, said aspirator tube comprising an insertion portion configured for insertion into the surgical workspace through a cannula, said aspirator tube having a distal end and a proximal end and a longitudinal axis, with an aspiration port disposed proximate the distal end;
a housing with a distal end fixed to the proximal end of the aspirator tube and a proximal end fixed to a suction tube in fluid communication with a vacuum source, with a portion of the suction tube disposed within the housing, and a suction control vent in the outer wall providing for fluid communication of ambient air through the suction control vent and into the suction tube when the suction control vent is unobstructed;
an LED light source disposed within the housing, said LED light source comprising and LED and an LED driver;
wherein:
the suction tube is in thermal contact with the LED light source; and
when the suction control vent is at least partially unobstructed and suction is applied through the suction tube, airflow through the suction tube cools LED and LED driver.

4. The aspirator tube of claim 2, further comprising:
a sheath disposed about portions of the light guide disposed about the aspirator tube.

5. The aspirator tube of claim 2, wherein:
the aspirator tube comprises an electrically conductive material, and electrically conductive portions of the aspirator tube distal to the light guide are covered with an electrical insulator, said electrical insulator terminating distally at a point proximal to the distal tip of the aspirator tube such that the distal tip of the aspirator tube is operable as a RF cautery electrode.

6. The aspirator tube of claim 5, wherein:
a portion of the aspirator tube at the proximal end of the aspirator tube is uncovered by the sheath and light guide, and thereby exposed to allow contact with an RF source.

7. The aspirator tool of claim 2, wherein the housing is configured as a handle for the aspirator tool.
